(19) United States
(12) Patent Application Publication
(10) Pub. No.: US 2020/0138372 A1
(43) Pub. Date: May 7, 2020

(54) SYSTEM AND METHOD FOR RECOGNITION OF MYOCARDIAL STUNNING

(71) Applicant: Intelomed, Inc., Warrendale, PA (US)

(72) Inventors: Anne Brumfield, Cranberry Township, PA (US); Christopher McIntyre, London (CA); Marcus Mianulli, Plymouth, MN (US)

(21) Appl. No.: 16/733,877

(22) Filed: Jan. 3, 2020

Related U.S. Application Data
(63) Continuation of application No. PCT/US2018/040964, filed on Jul. 5, 2018.
(60) Provisional application No. 62/528,768, filed on Jul. 5, 2017.

(51) Int. Cl. A61B 5/00; A61B 5/026; A61B 5/1455; A61B 5/024; A61B 5/021

(52) U.S. Cl. CPC A61B 5/4884; A61B 5/0261; A61B 5/746; A61B 5/021; A61B 5/4848; A61B 5/02416; A61B 5/7445; A61B 5/14551

(57) ABSTRACT

The invention provides early feedback to clinician or caregiver so that they may characterize the adequacy of the cardiorespiratory system over short-term time intervals. In that way, any deficiencies may be readily detected and treated before the patient's compensatory capacity is compromised. In one embodiment, a biological signal, having a waveform, is processed for deriving a perfusion parameter and a baseline value for the perfusion parameter. The invention monitors, in real time, the derived perfusion parameter and determines a variation of the computed perfusion parameter from the baseline value. The determined variation of the derived perfusion parameter is evaluated with respect to at least one variation threshold. A duration of time associated with the determined variation is measured that is reflective of the amount of time the derived perfusion parameter variation is above or below the at least one variation threshold. Based on the evaluation of the derived perfusion parameter with respect to the at least one variation threshold and the measured duration of time, a cardiovascular stress index is determined for the patient.

### CROSS-REFERENCE TO RELATED APPLICATION

This Application is a Continuation Application of International Patent Application No. PCT/US2018/040964 filed Jul. 5, 2018, which claimed the benefit of priority to U.S. Provisional Application 62/528,768, filed Jul. 5, 2017. The contents of the related Applications are hereby expressly incorporated by reference herein in their entireties.

### FIELD OF THE INVENTION

The invention is directed generally to the monitoring of perfusion conditions of a person, and more particularly to categorizing when a person is approaching a level of perfusion that is indicative of myocardial stunning which requires an intervention by a caregiver.

### BACKGROUND OF THE INVENTION

The circulatory flow of blood for delivering oxygen and nutrients to tissues and organs and for removing carbon dioxide, toxins and wastes therefrom is referred to as perfusion, and is an important factor in the health of a human being. Such a delivery and removal process is essential to maintaining cellular and tissue function, and overall health of tissues and organs. This circulatory delivery and removal of the blood depends upon the conditions of the lungs heart and blood vessels of an individual. Stress or other compromise in one system detrimentally affects the others, as well as overall circulatory function to achieve adequate perfusion.

Adequate blood flow to the tissues and organs must be maintained under varying forms and degrees of stress. Stress can be the aggregate impact of various internal or external physical, pathological, or environmental factors to which the body must respond in order to maintain physiological balance (homeostasis. In order to remain in what is considered a physiologically homeostatic state, the body must continuously adjust the various systems in order to meet constantly-changing demands. In a healthy patient, such adjustments will generally occur without a significant impact on the person. In other words, the person's cardiovascular and respiratory systems are able to compensate to these stressors and maintain homeostasis. However, in a situation where a person's ability to adjust is inadequate during a time of stress, the delivery of oxygen and nutrients to tissues and organs and the removal of carbon dioxide, toxins and wastes therefrom may be inadequate to meet cellular demands. Such a situation is referred to as decompensation. As a result, overall physiological function is compromised.

Such compromise may be the result of the ongoing failing health of a person, or may be associated with one or more acute stressful bodily insults. One such insult may be medical procedures or interventions the person is undergoing, often as a result of failing health, or some medical condition or ailment. As a result, perfusion decreases, or deficits may be indicative of the overall circulatory health of a patient. Perfusion deficits may result from a number of medical conditions, including hypertension, myocardial ischemic, cardiac dysfunction, hypovolemia, chronic obstructive pulmonary disease (COPD), pulmonary edema, anemia or apnea, for example. In other situations, a pronounced fall in perfusion may be brought on by a procedure that is undertaken for one or more medical reasons. Prolonged or repeated perfusion deficits to the heart muscle can result in a condition referred to as “cardiac or myocardial stunning”. Cardiac stunning is the reversible reduction of function of heart contraction.

One illustrative example of a patient medical intervention that may cause decreased perfusion and cardiac or myocardial stunning is a hemodialysis procedure. Generally, a person that has severe failing of their kidneys will undergo regular hemodialysis procedures that supplement or replace kidney function, that is the removal of waste products, the elimination of extra bodily fluid, and the restoration of electrolyte balance. Dialysis patients often have concomitant cardiovascuar disease. Such hemodialysis procedures are very stressful on the body, and require that already compromised cardiovascular and respiratory systems be able to respond to additional significant stress. As a result, such a procedure may create significant decreases in blood flow that leads to decompensation. That is, the person's cardiovascular and respiratory systems are unable to make adequate adjustments to maintain homeostasis. As a result, the patient may develop various symptoms (e.g., light headedness, short of breath, diaphoresis, chest discomfort, loss of consciousness) and clinical signs (e.g., drop in blood pressure, pulse irregularity, decreased pulse rate). Decompensation often goes unrecognized by a caregiver until it progresses to the point that the patient develops severe symptoms and signs. Diagnostic monitoring often reveals significant hypotension, cardiac rhythm disorders such as the onset of atrial fibrillation or ventricular tachycardia, and ECG changes indicative of myocardial ischemia (inadequate oxygen delivery to the heart muscle).

The symptoms and signs delineated above are often associated with pronounced falls in myocardial perfusion that may lead to the noted condition of myocardial stunning. Dialysis-induced recurring ischemia and repetitive myocardial stunning lead to the loss of adequate systolic (contractile) and diastolic (relaxation) cardiac function. It may also lead to chronic structural/functional changes in the heart and peripheral tissues, and has been shown to increase the risk of mortality. The ability to detect decreasing perfusion during dialysis prior to the development of patient symptoms and clinical signs would provide a window for caregiver medical intervention, and thereby the avoidance of a myocardial stunning condition with the patient

It has been well established that hemodialysis (HD) patients have significantly elevated rates of cardiac mortality, which are not driven by the same risk factors or pathophysiological processes that are important in the general population. Atherosclerotic coronary artery disease is not the predominant mode of death in patients receiving hemodialysis. Hemodialysis patients have been shown to develop progressive cardiac remodeling during long-term dialysis. Records from the US Renal Data System have shown that hemodialysis is an independent risk factor for the development of both de novo and recurrent heart failure. Furthermore, a significant percentage of cardiac mortality is due to sudden death, which has been shown to be temporally related to the dialysis procedure itself, i.e., the time period shortly after the dialysis treatment. Additionally, hemodialysis patients are more susceptible to developing myocardial ischemia during hemodialysis and ultrafiltration (UF) because of left ventricular hypertrophy, reduced peripheral arterial compliance, impaired microcirculation and ineffective vasoregulation.

Several studies have described the phenomenon of hemodialysis-induced myocardial stunning commonly encountered in hemodialysis patients, and the associated reductions in cardiac function Research has also shown that dialysis is associated with the development of malignant cardiac arrhythmias, and reduced survival. Measurement of myocardial blood flow during dialysis has demonstrated that hemodialysis can precipitate myocardial ischemia. Ongoing and recurrent episodes of ischemia precipitated by hemodialysis have negative consequences, leading to further myocardial injury and development of eventually nonviable myocardium. In addition, ischemia often causes deleterious changes in the electrical substrate of the heart, increasing the risk for malignant arrhythmias and sudden cardiac death. Echocardiography can be used to detect myocardial stunning during dialysis by identifying reductions in segmental and global systolic contractile function. Cardiac injury/stunning is associated with loss of cardiac function, increased cardiac events, and increased morbidity and mortality.

While there are various systems and methods that may be good markers of overall cardiovascular status resulting from long-term pathological and age-related changes, such systems only provide the status from chronic conditions and their associated outcomes. That is, those systems generally cannot characterize the functional adequacy of a cardiovascular system in the short term, such as acute changes noted during dialysis. Currently, the detection of myocardial stunning requires the use of serial imaging during dialysis to recognize the segmental reduction in systolic function. This is typically performed only for research purposes as it requires expensive equipment and highly skilled personnel, and is not a realistic diagnostic monitoring tool that can be utilized during routine patient care delivery. Furthermore, the identification of stunning using echocardiography assesses the presence of established myocardial stunning and is not useful to detect early myocardial stunning that allows for clinical interventions that may prevent organ hypo-perfusion within the hemodialysis treatment. As such, deficiencies in maintaining adequate tissue and organ perfusion is often not detected untilphysiological function is so compromised that the patient develops a decompensated state. Homeostasis cannot be maintained resulting in clinical instability and increased risk to the patient. This is typically performed only for research purposes and is not a realistic diagnostic tool that can be utilized for individual patient care delivery. Although, reduced perfusion is implied through regional reductions in contractile function, this has been robustly validated against direct measurement of intradialytic myocardial perfusion performed with both PET imaging and arterial spin labelled MRI perfusion.

As such, deficiencies in maintaining adequate tissue and organ perfusion is often not detected until physiological function is so compromised that the patient develops a decompensated state. Homeostasis cannot be maintained resulting in clinical instability and increased risk to the patient.

In the example of an ongoing stressful procedure, such as a hemodialysis, the treatment may have to be reduced or stopped completely. This prevents a patient from receiving the full benefits of such a procedure, resulting in the patient leaving the clinic in a fluid overloaded state that increases the risk for deleterious clinical events. Currently, there are no means to identify myocardial stunning resulting from hemodialysis-induced systemic circulatory stress early enough to allow timely intervention and modification of the dialysis procedure that allows safer delivery of hemodialysis treatment.

Accordingly, there is a need for systems and methods that are able to determine the presence of cardiovascular stress/stunning and recognize various conditions that are harmful to a patient early in the decompensation process. As such there is a need for such systems and devices that readily provide early feedback to a clinician or caregiver so that they may characterize the adequacy of the cardiovascular and respiratory systems over short-term time intervals, before the development of patient symptoms and clinical signs. In that way, any deficiencies may be readily detected and treated before the patient's compensatory capacity is compromised. The present invention addresses these and other needs in the art, as discussed herein.

### SUMMARY OF THE INVENTION

The invention provides systems and methods that are able to determine the conditions of cardiovascular stress and recognize various conditions that are harmful to a patient early in the decompensation process, so that the proper intervention can be determined and implemented prior to development of clinical signs and symptoms. It provides early feedback to a clinician or caregiver so that they may characterize the adequacy of the cardiovascular and respiratory systems over short-term time intervals. In that way, any deficiencies may be readily detected and treated before the patient's compensatory capacity is compromised. In one embodiment, a biological signal, having a waveform, is processed for deriving a perfusion parameter and a baseline value for the perfusion parameter. The invention monitors, in real time, the derived perfusion parameter and determines a variation of the computed perfusion parameter from the baseline value. The determined variation of the derived perfusion parameter is evaluated with respect to at least one variation threshold. A duration of time associated with the determined variation is measured that is reflective of the amount of time the derived perfusion parameter variation is above or below the at least one variation threshold. Based on the evaluation of the derived perfusion parameter with respect to the at least one variation threshold and the measured duration of time, a cardiovascular stress index is determined for the patient.

### BRIEF DESCRIPTION OF THE DRAWINGS

FIG. 1 is the overall flow summary for the evaluation of the Cardiovascular Stress Index (CSI).

FIG. 2A-F are graphical representations of determined cardiovascular parameters that provide CSI scores that are actionable in accordance with the invention and reflect abnormal and prolonged decreases in perfusion in patients with echocardiograms indicated myocardial stunning.

FIG. 3A-B are graphical representations of determined cardiovascular parameters that provide CSI scores that are not actionable in accordance with the invention and reflect normal perfusion levels in patients with echocardiograms negative for myocardial stunning.

FIG. 4 is a graph of an association between an actionable CSI score and reduction in pulse strength with features of cardiac stunning.

FIG. 5 is a graph of pulse strength during an episode of hypotension in dialysis procedures.

FIG. 6 is a schematic diagram of an exemplary hardware system for implementing embodiments of the invention.

FIG. 7 is a schematic diagram of another exemplary hardware system for implementing embodiments of the invention.

FIG. 8 is a schematic diagram of another exemplary hardware system for implementing embodiments of the invention.

FIG. 9 is a schematic diagram of another exemplary hardware system for implementing embodiments of the invention.

FIG. 10 is a schematic diagram of another exemplary hardware system for implementing embodiments of the invention.

FIG. 11 is a graphic design of signal waveforms used in accordance with an embodiment of the invention.

### DETAILED DESCRIPTION OF THE INVENTION

The present invention provides a way of recognizing a pronounced fall in cardiac perfusion or myocardial blood flow that may develop in a monitored person. Such cardiovascular insults may be due to failure in the overall cardiovascular and respiratory systems, or due to a stressful procedure or situation that a person is enduring, in which their body cannot adequately compensate. The invention does so by providing a unique analysis of biological waveforms. The invention uses a measure of perfusion indicated by the pulse amplitude or pulse strength of the waveform to detect changes detected from a person over time. The processing of the invention evaluates perfusion and its correlation to myocardial stunning. From such analysis, a unique index is developed providing a real-time and useable parameter reflective of the decreasing myocardial blood flow of a person so that a caregiver can determine an adequate intervention and the effects of such an intervention.

The present invention is directed to a system and method for recognizing circulatory stress, or rather, sustained decreases in cardiovascular function indicative of cardiac stunning in order to determine when a person is undergoing significant cardiac injury for the purposes of a suitable intervention. The present invention is further directed to providing a system and method for categorizing the level of circulatory stress prior to myocardial stunning in order to alert a medical caregiver that a particular person may require an intervention to avoid cardiac insult. The invention provides a readily-usable index for display and used by a caregiver, and for use in the identification of the cardiovascular stress and abnormal perfusion conditions that a person is undergoing.

The present invention also provides a unique way of analyzing and processing a biological signal to evaluate the cardiac components of that signal, and display alerts regarding the cardiovascular stress that may benefit from clinical intervention. As noted, both time-domain analysis and/or frequency-domain analysis are made on the biological signal for the evaluation of the stress and the display of usable, readily-available information related thereto. Specifically, various parameters are determined from a measured biological signal, and processed and combined in a unique fashion, in accordance with aspects of the invention, for providing useful information to the caregiver. The biological signal is acquired in a non-invasive manner, with few risks to a person. As discussed herein, various different sensors might be utilized for the purposes of the invention to provide the necessary biological signals for analysis.

Cardiovascular stress may be caused by a variety of different physical conditions, circumstances, and health conditions. As such, the present invention may be utilized to provide the detection of cardiovascular stress for a variety of different situations. In one use of the invention, a person having a health condition may be monitored, including those patients that may be undergoing some particular medical procedure or treatment to their body that may cause such cardiovascular stress. In another use of the invention, a healthy person may be monitored undergoing a physically stressful activity. Therefore, the invention is not limited to use with a healthcare patient, or an unhealthy person per se.

For example, various different procedures might be performed on a patient to address other health issues of the patient that are not specifically or directly related to the respiratory and/or cardiovascular systems, but which may have an overall impact on such systems. One such procedure is a hemodialysis procedure that is utilized with patients that are experiencing kidney failure. For example, as blood and other fluids are removed from a patient for the hemodialysis filtering process, a significant amount of cardiovascular stress may be created during the dialysis process. If such stress is not recognized early enough to allow for a suitable intervention, the entire dialysis process may need to be altered or completely stopped for a more vigorous medical intervention with the patient. The present invention, however, is not limited to dialysis processes, and thus, can be utilized for detecting cardiovascular stress in patients undergoing other procedures or are being treated for other clinical conditions. Also, as noted, it may be used with generally healthy patients that may experience particular conditions that create cardiovascular stress that then requires some particular intervention from the person themselves or by a caregiver who is monitoring the person. However, for illustrative purposes and discussion herein, a person on which the invention is used is referred to as a “patient”, and person using the information is referred to as a “caregiver”, although those terms are not limiting with respect to the invention.

The present invention provides a unique analysis, utilizing baseline values and subsequent changes in values of perfusion from baseline, indicated by changing pulse strength or pulse amplitude which may be derived in the frequency-domain and/or time-domain of a biological signal. This determination along with is used to provide a cardiovascular stress index (CSI) parameter that reflects a stress condition and that can be used to diagnose one or more cardiovascular conditions. Such an index parameter and results of the invention can be compared against traditional echocardiography methods and measurements to correlate the CSI parameter with actual cardiac stunning and other conditions.

In accordance with one embodiment of the invention, the biological signal utilized is a photoplethysmogram signal, also referred to as a “pleth” signal or PPG signal. The PPG signal is a biological signal optically obtained using a sensor similar to that used for obtaining a pulse oximetry reading. The obtained biological signal, such as the PPG signal, is then processed in accordance with the present invention utilizing time-domain analysis and/or frequency-domain analysis of the biological signal in order to evaluate and characterize decreasing perfusion levels. Although a PPG signal is used in one embodiment of the invention, other biological signals might also be used to reflect cardiovascular components for the analysis, and so the invention is not limited to the use of just a PPG signal.

The signal is processed utilizing a computer or other suitable processing system or device, such as a processing system provided by a mobile device (e.g., a pad device or smart phone). However, other computer systems and processing systems may be implemented, and thus, the invention is not limited to the specific device or system from which the invention may be implemented.

In one embodiment of the invention, parameters such as Pulse Strength (PS) or pulse amplitude are utilized and are extracted from a measured PPG signal. Such extraction of a PPG signal and waveform and measurement of pulse strength is set forth in various examples in U.S. Patent Nos. and patent application Ser. Nos. 8,423,108; 9,002,440; 9,172,579; 9,808,160; 14/830,344; 14/295,856; 14/302,411, which are incorporated herein by reference in their entireties.

One suitable device for implementing the invention is the CVINSIGHT® system available from Intelomed, Inc. of Cranberry Township, Pa. The CVINSIGHT® Patient Monitoring & Informatics System is implemented on a tablet device having a suitable touch screen and display for the purposes of displaying the outputs of the invention and the specific cardiovascular stress index and diagnosis, as discussed herein.

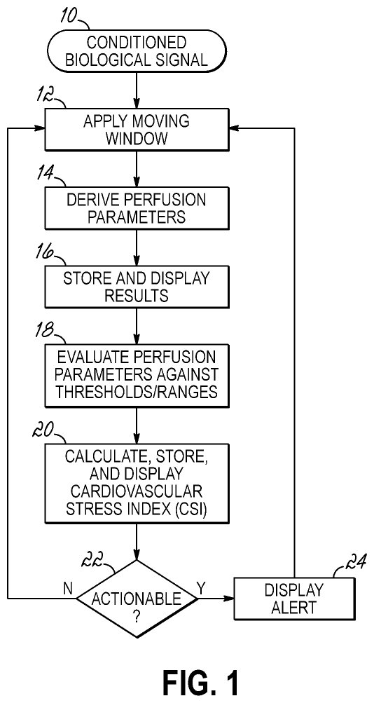

Turning to FIG. 1, a flowchart is illustrated for the purposes of explaining one embodiment of the invention. Such program flow and program flow paths will be executed in suitable software in a processing device or system, as discussed herein. While the program flow is illustrated in the particular arrangement for explanation and discussion of the invention, the invention is not limited to a specific order for each of the steps or a specific hardware and software environment, and thus, some deviation may be utilized in the processing of the biological signal, as disclosed herein.

Turning to FIG. 1, the conditioned PPG signal is obtained from a suitable sensor (block 10). Various usable sensors for obtaining biological signals that may be useful for the invention, such as a PPG signal, are set forth in Table 4 below as discussed. The sensor may be non-invasively attached to the patient, such as on the forehead of a patient. The sensor obtains the necessary biological signal as illustrated in block 10. The measured biological signal is conditioned and filtered as is appropriate for further signal processing. In another embodiment of the inventions, a non-contact sensor, such as a photo image might be used to extract a biological signal, such as a PPG signal.

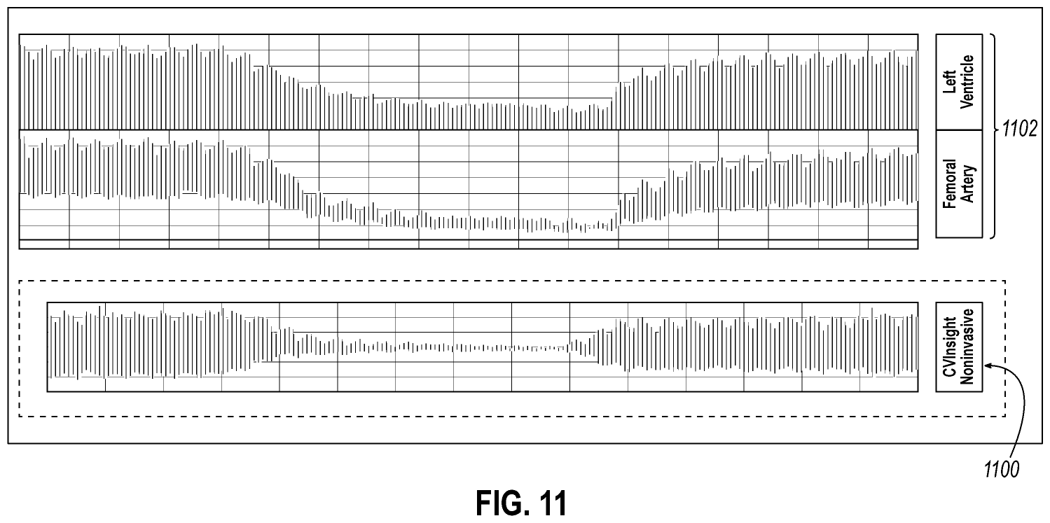

The present invention takes advantage of a noninvasive sensor for obtaining biological signals. Referring to FIG. 11, a contact sensor, as discussed herein is utilized such as a sensor that utilizes infrared light that may then be absorbed by the blood proportionally to the blood volume. The remainder is reflected from the arteriole bed back to the sensor. The signal will then be processed as discussed herein and may be displayed as a waveform as illustrated at 1100 in FIG. 11. The noninvasive sensor utilized in one embodiment of the invention has been shown to closely mirror wave forms associated with more invasive hemodynamic measurements, such as illustrated at 1102 in FIG. 11 which is indicative of a wave recording made during mechanical manipulation of pre-load with a balloon catheter placed in the superior vena. Other sensors as noted may be used to provide the biological signal.

For processing purposes, a moving processing window is applied, the signal that is acquired, as shown in block 12. In one embodiment of the invention, a thirty-second moving window may be a suitable size to apply to the streaming waveform data of the biological signal, but the window size is variable and the invention is limited to the noted example.

In accordance with one aspect of the invention, perfusion parameters such as pulse strength or pulse amplitude might be utilized and derived from the biological signal (block 14). In one embodiment, a pulse amplitude is derived from the PPG signal. Pulse amplitude is reflected in an evaluation in the time-domain, and is thus derived using time-domain analysis (e.g. peak and valley detection, area-under-the-curve evaluation, etc). In another embodiment, pulse strength is used and is derived from frequency-domain analysis of the PPG signal. For example, a Fourier process might be performed on the acquired time-domain signal for the frequency analysis to derive pulse strength. Pulse strength, in one embodiment, is considered the power of the signal at the pulse rate frequency. That measured power might take into account the power at the fundamental frequency. Alternatively, it might take into account the power at the fundamental frequency and one or more harmonic frequencies.

The various derived values or levels of the perfusion parameters are stored (block 16) and can be derived as raw values or as a normalized value from a baseline. In one embodiment of the invention, the perfusion parameter will be derived and will be monitored as a percent change of a pulse strength or pulse amplitude from a measured baseline value. Accordingly, before a procedure, such as hemodialysis is started, the biological signal is acquired to have or determine a baseline value of pulse amplitude or pulse strength that is then used in determining the cardiovascular stress index of the invention.

The derived perfusion parameter is then monitored in real time for determining a variation of the computed perfusion parameter from the baseline value. Perfusion parameters are then evaluated against ranges and thresholds (block 18) associated with the parameter. In one exemplary embodiment, the perfusion parameter might be normalized from a baseline pulse strength or pulse amplitude value. Then, the variation determined from the baseline might be determined and reflected as a percentage variation from the baseline. The derived parameter and the determined variation is evaluated against at least one variation threshold. For example, a percentage variation of the perfusion parameter might be evaluated against a threshold percentage or against a range having certain thresholds as endpoints. In one embodiment, the perfusion parameter is evaluated against the noted range or noted threshold value.

In accordance with another feature of the invention, the duration of time of the determined variation of the perfusion parameter within the range or above or below the noted threshold is also measured and determined. For example, while the perfusion parameter variation may occur for short periods of time that does not indicate an actionable degradation in cardiovascular health of a patient, the invention evaluates duration length value for use in the overall determination of cardiovascular health. In accordance with one feature of the invention, the measured perfusion parameter and its relation to a threshold or range is combined with a duration value for that perfusion parameter to determine a cardiovascular stress index (CSI). That is, based on the evaluation of the derived perfusion parameter with respect to the at least one variation threshold and the measured duration of time a cardiovascular stress index is determined for the patient.

Multiple alert thresholds and ranges and time durations are continuously evaluated to determine a cardiovascular stress index (CSI). In accordance with one feature of the invention, the perfusion parameter level is determined and is then compared against multiple ranges or thresholds. Also, the time duration of the perfusion parameter being above or below a particular threshold or within a range is evaluated to calculate the CSI score or value. Depending on how the perfusion parameter is characterized, the perfusion parameter might be above or below a defined threshold value or within a range. The combination of a particular perfusion parameter level with respect to a threshold or range as measured for a sustained amount of time will result in a calculation of the cardiovascular stress index (CSI). The CSI score or value is displayed or reported to caregiver as a particular alert level and may be used by a caregiver to take action.

For example, a score of 0 might be considered measured perfusion parameter associated with a normal perfusion parameter level or value. That is, as long as the measured perfusion parameter stays within a range or is only negligibly below or above a threshold associated with the baseline value, there would essentially be no alert level or message for that score. Other measured perfusion parameter levels at a sustained amount of time might lead to other CSI scores and appropriate alerts or messages.

In one embodiment of the invention, the perfusion parameter of pulse strength is evaluated and specifically, thresholds or ranges noting a drop in value or decreased value of the derived perfusion parameter from the baseline value are used. Based on the evaluation of the derived perfusion parameter with respect to variation threshold and the measured duration of time a cardiovascular stress index for the patient is determined. That is, the determined variation, and the time duration of that determined variation with respect to the threshold are used for determining the cardiovascular stress index and yielding a CSI score. More specifically, the measured duration of time associated with the determined variation is compared against a threshold and that measured duration of time against the threshold is used for determining a cardiovascular stress index for the patient. In one embodiment of the invention, duration thresholds of 30 minutes, 60 minutes, 120 minutes, and 180 minutes are used.

For example, if a measured perfusion parameter level is below a −20% threshold (that is 20% below a measured baseline for pulse strength or pulse amplitude) and that level is sustained for greater than 30 minutes, a CSI score of 1 might be calculated and displayed as an actionable alert level of 1 for the caregiver. In accordance with one feature of the invention, a CSI score of 1 or above is considered actionable and an indication of myocardial stunning. Beyond that CSI score of 1, more sustained decreases below other variation thresholds and/or further durations of time at that variation beyond other time thresholds might yield other scores. For example, a CSI score and alert level of 2 is set at a perfusion parameter level that is below a −40% threshold from the baseline value and that is sustained for a duration greater than 60 minutes. Similarly, a CSI score and alert level of 3 is set at a measured perfusion parameter level that is below a −40% threshold from the baseline value and that is sustained for a duration greater than 120 minutes. A CSI score and alert level of 4 is set at a measured perfusion parameter level that is below a −40% threshold from the baseline value that is sustained for a duration greater than 180 minutes. Therefore, in accordance with one aspect of the invention, the cardiovascular stress index or CSI values may be determined based upon the measured and derived perfusion parameter level and its variation with respect to the at least one variation threshold and evaluation of the duration of time at such variation from a baseline value to determine the risk and onset of myocardial stunning. The CSI score can be stored and displayed.

As may be appreciated, various thresholds may be set as end points for certain ranges for the measured perfusion parameter. Then, if a perfusion parameter falls in that range (e.g.−40%≤×≤−20%) for some duration of time, (e.g. ≥30 minutes), a CSI score might be determined. Increased time durations for the perfusion parameter variation that are spent in that range might result in calculations of ever increasing CSI score and appropriate alert levels. Still further, a value within a different range (e.g. −60%≤×≤−40%) for a selected duration, such as ≥30 minutes, might yield a calculated CSI score that is higher than a measured perfusion parameter value at the same or longer duration in a lower range. The CSI values from 0-4, with coinciding alerts, are displayed for a caregiver for the purposes of possible intervention with a patient. Generally, as noted, a score of 1-4 is actionable for indicating a situation of myocardial stunning.

In accordance with one embodiment of the invention, certain CSI scores are considered indicative of a patient condition that may require intervention by a caregiver. As such, certain scores are considered actionable for a caregiver (block 22). For example, while a CSI score between 1-2 may be watched but not acted upon by a caregiver, if the CSI score is 3-4, the caregiver will be alerted to act. For actionable values and results, an alert condition and/or message is displayed appropriately for a caregiver, so they know to intervene with a series of actions or a particular solution for the patient. (block 24). In one embodiment, just the CSI score is displayed. In other embodiments, possible interventions, as noted herein, might be displayed for guidance to a caregiver to address the situation. For example, repositioning, fluid IV's and other intervention steps might be displayed.

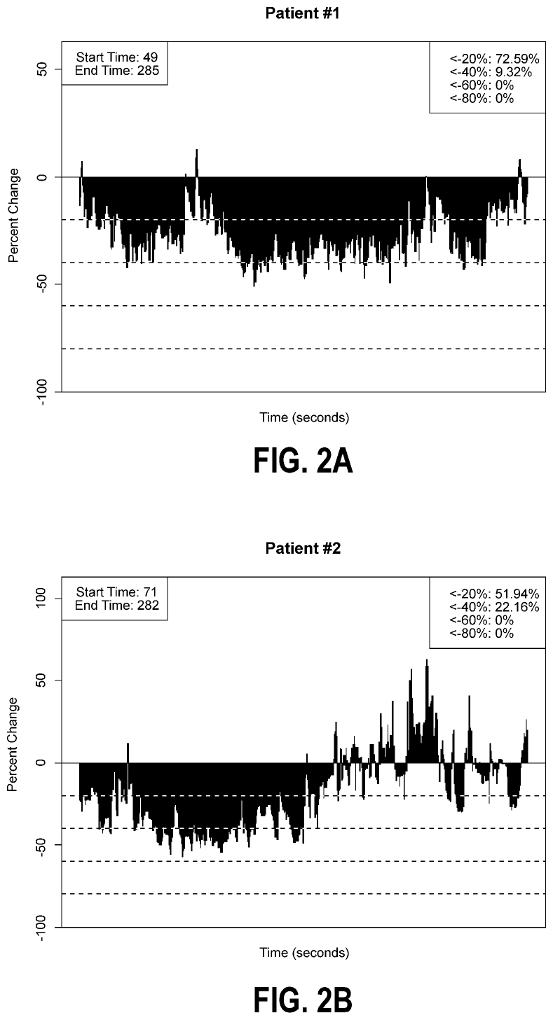

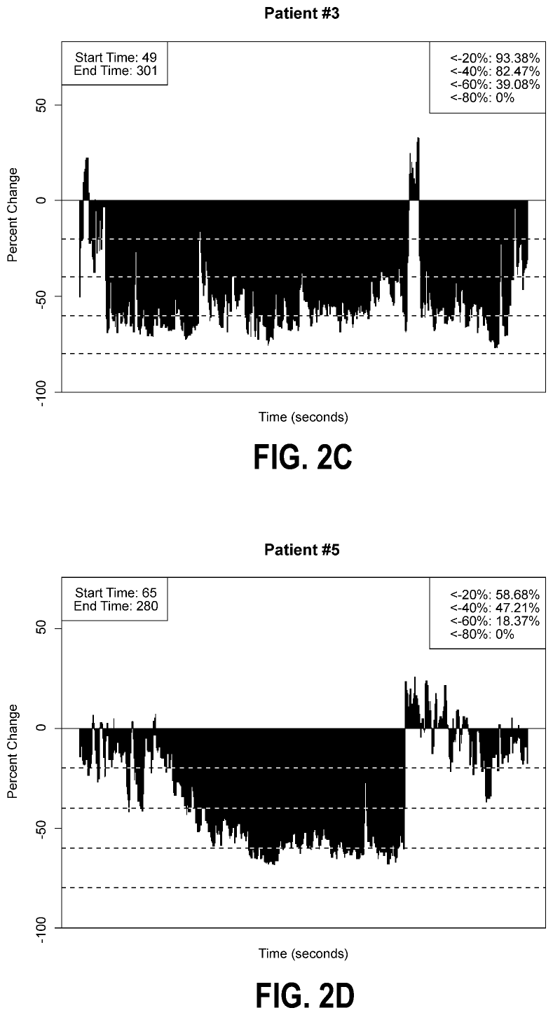

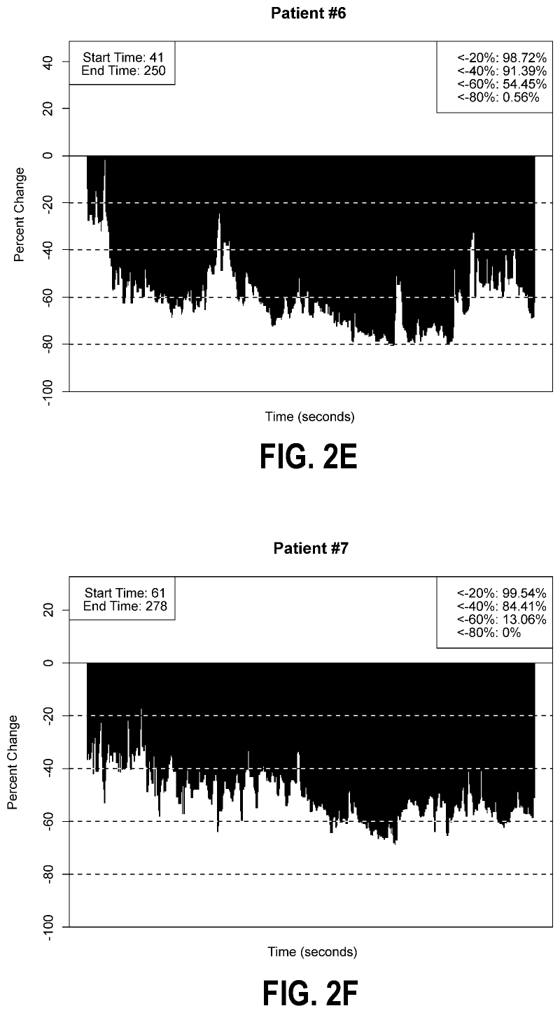

In accordance with one aspect of the invention, various patients were monitored during hemodialysis and their perfusion parameter values were measured for calculation of a CSI score. FIGS. 2A-F illustrate graphs of the normalized perfusion parameter levels (pulse strength) for various patients and the variation of such levels from a baseline value. Such patients were evaluated in accordance with the invention and CSI scores calculated. Those that exhibited CSI scores above 0 in accordance with the invention were also evaluated as discussed below with cardiovascular physiologic monitoring to confirm that all of them exhibited myocardial stunning conditions with significant reductions in pulse strength for most of the session of evaluation. Such actionable CSI scores were thus correlated to myocardial stunning to provide real time and actionable values that may be used by a caregiver.

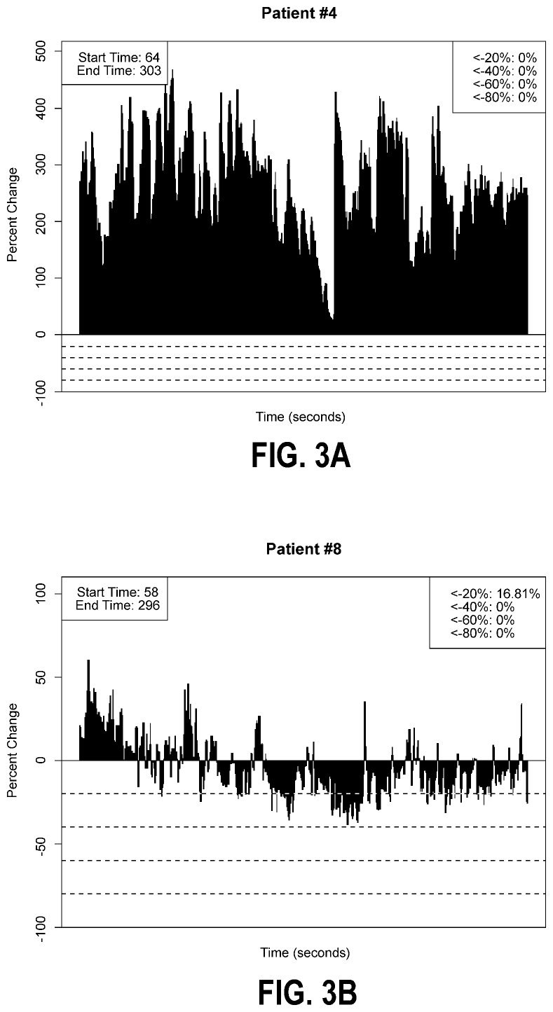

Alternatively, FIGS. 3A-B illustrate the normalized measured perfusion parameters of patients who were also evaluated in accordance with the invention. Both patients had no actionable CSI scores (e.g. Score=0) or significant reduction in pulse strength, nor evidence of myocardial stunning. As such, the efficacy of the invention was confirmed through CV physiologic monitoring that is correlated to the actionable and non-actionable CSI values.

For the measurements, dialysis treatments were delivered in a single location by a single operator. Hemodialysis was delivered using a Fresenius 5008 system available from Fresenius SE 8 Co of Bad Homburg, Germany. High flux polysulfone dialyzers were used as per the patient's usual prescription. Treatment times were either 3.5 or 4 hours. Dialysate was programmed according to each patient's individual prescriptions; sodium of 140 mmol/L for all patients except one who was 138 mmol/L, 4 patients had 1.5 mmol/L potassium dialysate, and 4 had 3.0 mmol/L, all patients had a dialysate calcium of 1.25 mmol/L and bicarbonate ranged from 34 to 40 mmol/L. Anticoagulation was achieved using low-molecular weight heparin (dalteparin) except for one patient who received unfractionated heparin. Dialysate flow was 500 mL/min and temperature was set at 36.5° C. For each session, net ultrafiltration was set on an individual basis according to ideal dry weight. Blood pump speed varied between 320 and 400 mL/min. Dialysis access was via arteriovenous fistula in 5 patients and via central venous catheter in the remaining 3 patients. All studies were conducted midweek after a 48-hour interdialytic period. For this study, intradialytic hypotension (IDH) was defined as a fall in systolic blood pressure (SBP)≥20 mgHg and/or ≤100 mg/Hg in association with typical symptoms of hypotension including, nausea, light-headedness, or cramping requiring intervention.

For the measurements, and continuous cutaneous perfusion monitoring, a sensor was placed to the right of the patient's forehead midline, approximately 2.5 cm above the level of the nose. The sensor consisted of a reusable pulse oximetry sensor and disposable adhesive carrier. For the illustrated example, a CVI SensorCircle™ Calibrated Cap carrier was used. Once attached, a baseline recording was captured to ensure proper sensor placement and adequate PPG signal prior to initiating hemodialysis. Hemodynamic data was continuously captured from 20 minutes prior to the hemodialysis treatment until 30 minutes after the end of dialysis. Data was recorded, but not reviewed until after the treatment had concluded. All acquired data was analyzed post hoc using LabVIEW from National Instruments of Austin, Tex., USA. Events were annotated on the monitoring device for each individual patient, including: initiation and completion of echocardiogram, initiation and completion of hemodialysis treatment, any symptoms experienced by the patient including dizziness, lightheadedness, cramping, nausea, headache, pain/discomfort, decrease in blood pressure, or any interventions delivered by the care provider such as ultrafiltration changes, position change, or bolus of NaCl.

For correlation to myocardial stunning events associated with the measured CSI scores, Echocardiography was performed by a trained investigator prior to commencing and 15 minutes before the end of hemodialysis using commercially available equipment (1.5-3.6 MHz M4S probe, Vivid-q, GE Medical Systems, Soningen, Germany). Standard parasternal long-axis, short-axis and apical and parasternal 2 and 4 chamber views were recorded for off-line digital analysis with a semi-automated computer program (EchoPac, GE Healthcare) using 2D speckle tracking software. Images were anonymized and analyzed in a random order by an investigator and a random sample of these images were analyzed in random order by a second appropriately trained investigator to determine estimates of inter-observer reliability. Three cardiac cycles were analyzed for each time point and values derived for segmental (12 left ventricular segments) and global longitudinal strain. If a myocardial segment underwent a reduction in longitudinal strain greater than 20%, it was defined as a regional wall motion abnormality (RWMA). The presence of myocardial stunning was defined as the presence of 2 or more RWMAs in the left ventricle. The number of left ventricular, segments exhibiting a reduction in longitudinal strain of more than 20% were also recorded.

Statistical analysis was performed using SPSS Statistics version 23 from BM, Chicago, Ill., USA. Continuous variables are expressed as mean±SD. All data were tested for normality using the Shapiro-Wilk test. Comparison of continuous outcomes between 2 groups was performed using the independent t test for parametric data and Mann-Whitney U test for nonparametric data. Comparisons of related outcomes at two different time points were performed using the paired t test for parametric data and the Wilcoxon signed-rank test for nonparametric data. Bivariate correlation was assessed using Pearson's correlation coefficient for parametric data and Spearman's coefficient for nonparametric data. An alpha error of less than 5% (P<0.05) was statistically significant.

Baseline clinical characteristics of the various patient subjects are shown in (Table I). Subjects mean age was 59.1±13.3.3 years. Six patients were on a thrice weekly dialysis regimen, 2 dialyzed 6 days per week. Of these patients, 2 were female and 6 were male. Dialysis vintage mean was 72±66.8 months ranging from 10 to 180 months with a hemodialysis vintage mean of 60±61.7 months ranging from 5 to 158 months. The most common causes of end stage renal disease were hypertension (25%) and diabetes (25%). Other causes included lithium toxicity, thrombotic thrombocytopenic purpura, obstruction, and IgA Nephropathy. Comorbidities included hypertension (88%), diabetes (50%), coronary artery disease (38%), and congestive heart failure (25%). Total ultrafiltration 1827.5±823.7 mL, with one patient having no fluid removed due to hypotension preceding treatment, max ultrafiltration rate range was 0 to 858 mL/min. All patients were taking either monotherapy or combination antihypertensive/cardiac medication, (Table I). Intradialytic symptoms, yet not necessarily associated with intradialytic hypotension included cramping (2 patients, without IDH), nausea (2 patients with IDH), headache (1 patient, without IDH) (Table 2).

All but one patient displayed a reduction pulse strength of up to 20%. That lasted for, 157.9±69.5 minutes (range 40.5-235.3), which corresponded to, 69.8%±30.1% (range 16.9-99.6) of the hemodialysis session, with onset at <1 to 43.3 minutes. Six patients had a greater than 40% reduction in pulse strength lasting for 107.5±86.7 minutes on average, or 47.8%±38.1% of their total hemodialysis treatment session. The reduction in pulse strength ≥40% was observed at 11.4±10 minutes into hemodialysis treatment. Pulse strength improved in one symptomatic patient throughout HD after a 0.9% NaCl bolus and without ultrafiltration during treatment; it is worth noting that this patient's baseline was atypical as he had taken his prescribed antihypertensive medication prior to the commencement of HD due to dialysis time change for this study.

TABLE 1
Demographic information and medical history of patient subjects

| | |
|---|---|
| Age | 59.1 ± 13.3 |
| Gender (WF) | 6/2 |
| Diabetes (Y/N) | 4/4 |
| History of CHF (Y/N) | 2/6 |
| History of CAD (Y/N) | 3/5 |
| ACEi/ARB (YIN) | 4/4 |
| Beta blocker (YIN) | 5/3 |
| Statin therapy (Y/N) | 3/5 |
| Duration of HD (months) | 72 ± 66.8 |
| Previous transplant (Y/N) | 3/5 |
| Systolic BP (mmHg) | 151 ± 16.3 |
| Diastolic BP (mmHg) | 71.1 ± 30.4 |
| Hemoglobin (g/L) | 110.6 ± 26.2 |
| Thrombocyte count | 175.9 ± 81.6 |
| Urea (mmol/L) | 19.8 ± 3.69 |
| Creatinine (mmol/L) | 778 ± 95.2 |
| Sodium (mmol/L) | 135 ± 4.64 |
| Albumin (g/L) | 38.2 ± 2.66 |

All biological parameters represented as pre-dialysis values.
ACEi = angiotensinconverting enzyme inhibitor;
ARB = angiotensin receptor blocker;
BP = blood pressure;
CAD = coronary artery disease;
CHF = chronic heart failure;
HD = hemodialysis.

Continuous cardiovascular physiologic monitoring was performed throughout the dialysis session using oximeter-based pulse waveform analysis in accordance with the invention, such as using a [CVInsight® Monitoring System, from Intelomed, Inc.]. Longitudinal strain (LS) values for 12 left ventricular segments were generated using speckle-tracking software [EchoPac, GE], to assess the presence of hemodialysis-induced regional wall motion abnormalities (RWMA), indicative of myocardial stunning.

From a review of the cardiovascular monitoring in relation to various CSI scores, the invention established that actionable CSI scores determined in accordance with the invention reflected actual myocardial stunning in the patient.

A summary of these findings is represented in Table 2 and 3 below.

TABLE 2

| Subject ID | Tx Start | Tx End | PS < −20% | PS < −40% | PS < −60% | PS < −80% | ECHO Stunning | CSI Score |
|---|---|---|---|---|---|---|---|---|
| 1 | 49 | 285 | 73% | 9% | 0% | 0% | Yes | 1 |
| 2 | 71 | 282 | 52% | 22% | 0% | 0% | Yes | 1 |
| 3 | 49 | 301 | 93% | 82% | 39% | 0% | Yes | 4 |
| 4 | 64 | 303 | 0% | 0% | 0% | 0% | NO | 0 |
| 5 | 65 | 280 | 59% | 47% | 18% | 0% | Yes | 2 |
| 6 | 41 | 250 | 99% | 91% | 54% | 1% | Yes | 4 |
| 7 | 61 | 278 | 99% | 84% | 13% | 0% | Yes | 4 |
| 8 | 58 | 296 | 17% | 0% | 0% | 0% | NO | 0 |

Tx = Treatment
PS = Pulse Strength (normalized to percent change)

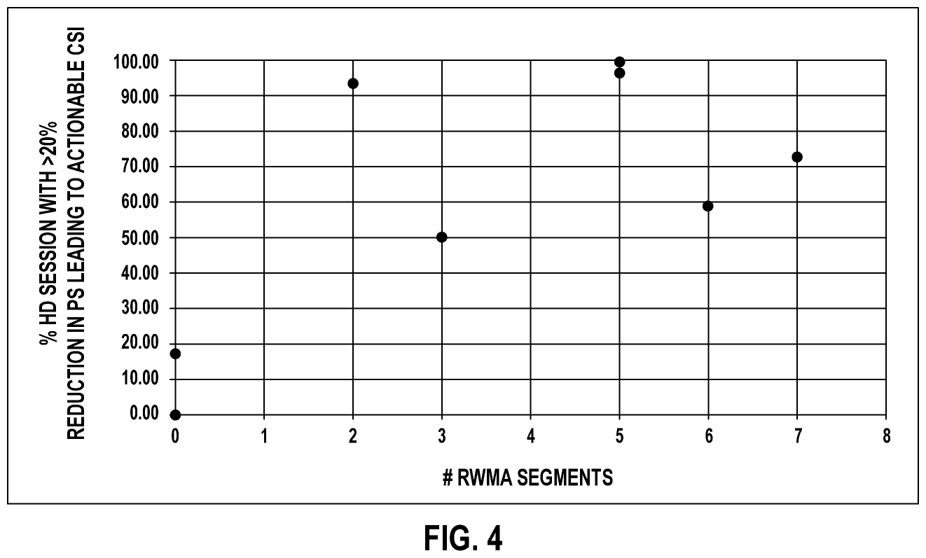

Referring to FIG. 2A-2F, various of the patients' changes in pulse strength as reflected in actionable CSI scores are illustrated in accordance with the invention. Six out of eight patients exhibited myocardial stunning. The number of RWMA segments affected ranged between 2 and 7 as illustrated in FIG. 4. Real-time measurable markers of circulatory stress included systolic blood pressure monitoring and blood volume measurements with a minimum blood volume of 88.6%±2.58. Two patients had a systolic blood pressure <100 mmHg with symptoms of intradialytic hypotension requiring intervention, of which stunning was evident in one, 5 patients had a systolic blood pressure reduction of 20 mmHg, with stunning in three, I patient had a further reduction of 40 mmHg and showed stunning, note that this patient is also captured in the previous 2 categories. There was not a consistent relationship between BP reduction and myocardial stunning, nor blood pressure reduction and pulse strength reduction. So, the determined CSI score were the indication of myocardial stunning.

All patients that demonstrate an actionable CSI score, such as a reduction in cutaneous PS≥40%, also exhibited myocardial stunning (P=0.005). whereas the 2 patients did not exhibit stunning showed no actionable CSI score, or reduction of pulse strength or regional wall motion abnormalities. FIG. 4 shows a relationship between actionable CSI scores in accordance with the invention as reflected by the amount of time in a hemodialysis session with a reduced pulse strength and regional wall motion abnormalities. A reduction in pulse strength was observed in all subjects who developed signs and symptoms of IDH during their treatment, furthermore pulse strength changes were noted to rapidly reflect the hemodynamic effects from clinical interventions for these episodes as illustrated in the plots of FIGS. 2A-2F and 3A-3B. These patients also exhibited SpO₂ variably and/or pulse irregularity. There was an association between the number of myocardial segments that underwent >30% reduction in LS and the duration of ≥40% reduction in PS (P=0.04, r=0.73).

FIG. 2A illustrates a patient having an actionable CSI score. Based upon the change in the pulse strength as indicated on the y-axis over a period of time, the patient would have a CSI score of 1 in accordance with the invention.

As illustrated in FIG. 2B, patient number two, although exhibiting a greater percent change in pulse strength, also has a CSI value or score of 1, based upon the shorter duration of the lower pulse strength over the measurement cycle.

Patient number three illustrates a significant reduction in the pulse strength from a baseline or in the reduction extended over significant amount of time within the measurement cycle. As such, patient number three exhibited a CSI score of 4 based upon the graph as illustrated in FIG. 2C.

FIG. 2D illustrates measurements for patient number five. Although patient number five shows a significant reduction in the pulse strength at amounts that are close to the significant changes from baseline illustrated in FIG. 2C for patient number three, the duration of significantly shorter and thus reflected a CSI score of two.

Patients six and seven, as illustrated in FIGS. 2E and 2F, both illustrated significant changes in the pulse strength from a baseline value for significant durations of time, and actually show a significant pulse strength drop over most of the duration of the cycle. Patients six and seven had CSI scores of 4 each.

FIGS. 3A and 3B, illustrate measurements for patients four and eight wherein there was no significant change in pulse strength as illustrated FIG. 4, or only reductions for shorter durations of time. Thus, the CSI scores for both are 0, and thus not actionable. Patients one-three, five-seven all had actionable CSI scores indicative of myocardial stunning as discussed herein.

There was no correlation between reduction in pulse strength and age (P=0.56, r=0.695). There was also no correlation between pulse strength reduction ≥40% and systolic blood pressure <100 mmHg (P=0.60, r=0.218), symptoms of IDH (P=0.73, r=−0.149), minimal blood volume (P=0.87, r=0.069), total UF volume (P=0.33, r=0.395), nor HD vintage (P=0.50, r=0.281) (Table 2).

TABLE 3
Hemodialysis information and symptoms of study participants

| Participant | UF Total | Mean UFR | Min BV | Lowest SBP | IDH | Cramping | Headache | Nausea |
|---|---|---|---|---|---|---|---|---|
| 1 | 2000 | 522 | 91.6 | 148 | no | no | no | no |
| 2 | 2070 | 650 | 88.9 | 94 | yes | no | no | yes |
| 3 | 1950 | 600 | 89 | 111 | no | no | no | no |
| 4 | 0 | 181 | 89.9 | 100 | yes | no | no | yes |
| 5 | 2500 | 321 | 87.8 | 143 | no | yes | no | no |
| 6 | 2100 | 627 | 83.7 | 126 | no | no | yes | no |
| 7 | 1400 | 400 | 91.4 | 123 | no | no | no | no |
| 8 | 2600 | 650 | 86.8 | 120 | no | yes | no | no |

BP = blood pressure; BV = blood volume; IDH = intradialytic hypotension.; UF = ultrafiltration; UFR = ultrafiltration rate.

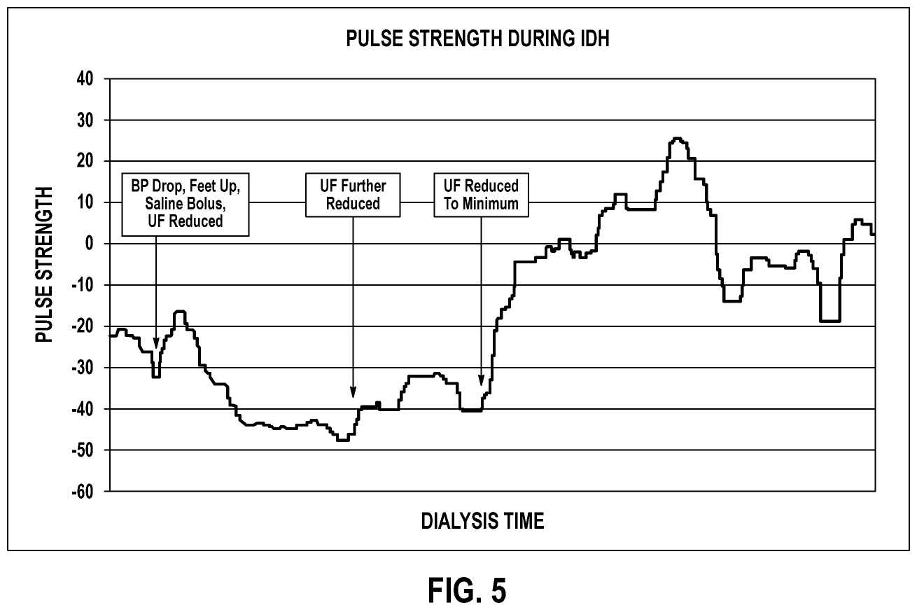

FIG. 5 illustrates a pulse strength response during an episode of symptomatic intradialytic hypotension in which the subject's pulse rate reduced quickly at the beginning of dialysis, with the development of IDH symptoms (nausea, dizziness), SBP decreased by 25 mmHg (100/53) within 15 minutes of treatment initiation. At that time, ultrafiltration was reduced, patient was placed in Trendelenburg position and a 100 mL bolus of 0.9% NaCl was administered to reduce symptoms of hypotension. The pulse strength rapidly to intervention by increasing cardiac preload and responded even further once ultrafiltration rate was reduced to minimum (63 mL/h). Such interventions might be addressed upon an indication of an actionable CSI score.

The measured CSI effectively identified hemodialysis patients with hemodialysis-associated myocardial stunning, potentially enabling interventions aimed at preventing myocardial injury. This is particularly relevant as there was no correlation between myocardial stunning and conventionally utilized indices of intradialytic hemodynamic stability such as blood pressure reduction, symptoms of intradialytic hypotension or reduction in blood volume.

Myocardial stunning is indicative of compromised myocardial perfusion. The presence of myocardial stunning is associated with increased mortality, as its recurrent injury pattern during chronic hemodialysis has a cumulative effect on cardiac function. Thus, it is important to intervene to prevent the progression of cardiac dysfunction using the actionable CSI scores.

It has been demonstrated that currently used indicators of dialysis-related circulatory stress are not adequate for predicting myocardial stunning, and often occur at a point that is too late to intervene/prevent injury. Therefore, having the invention that sensitively detects the propensity for cardiac injury and does so within a temporal window for dialysis-based intervention is valuable to improve patient outcomes.

Importantly, commonly used methods for the monitoring of intradialytic hemodynamic stability failed to identify those patients with either myocardial stunning or a significant reduction in pulse rate. This is likely because the development of intradialytic hypotension and related symptoms occur late during cardiovascular stress, when the patients' compensatory responses are no longer able to maintain cardiovascular stability.

The invention addresses the necessity for individualized care and the inquiry into individual pathophysiology of hemodialysis-induced cardiovascular stress. Because pulse strength reduction occurred early into hemodialysis treatment, there is a need to acquire more specific information to identify possible interventions for the reduction of such significant decline in the pulse strength, reducing the cardiovascular effects that conventional treatment regimens can cause. Modifications to the hemodialysis process to improve the hemodynamic tolerability have been shown to reduce the evidence of dialysis-induced cardiac injury lowering the rate of cardiovascular morbidity and mortality in the dialysis population.

Using the CSI score of the invention, action may be taken sooner for a patient. A variety of innovative approaches both dialysis-base (dialysate cooling, HD biofeedback, longer and or more frequent treatment times, individual care planning-positioning during treatment, UFR modifications), and non-dialysis based (review of antihypertensive medications, intensive glycemic control, dietary sodium management to reduce water intake) have proven effective in the reduction of myocardial stunning, cardiovascular stress, intradialytic hypotension, and improved treatment outcomes.

Accordingly, a CVI score using pulse strength via percutaneous perfusion monitoring correlated with echocardiographic evidence of myocardial stunning provides a useful tool for addressing hemodialysis issues. Therefore, the cardiovascular invention has the potential to identify cardiovascular stress earlier than traditional markers of hemodynamic stability during hemodialysis and can provide a window of opportunity for intervention prior to the occurrence of cardia injury.

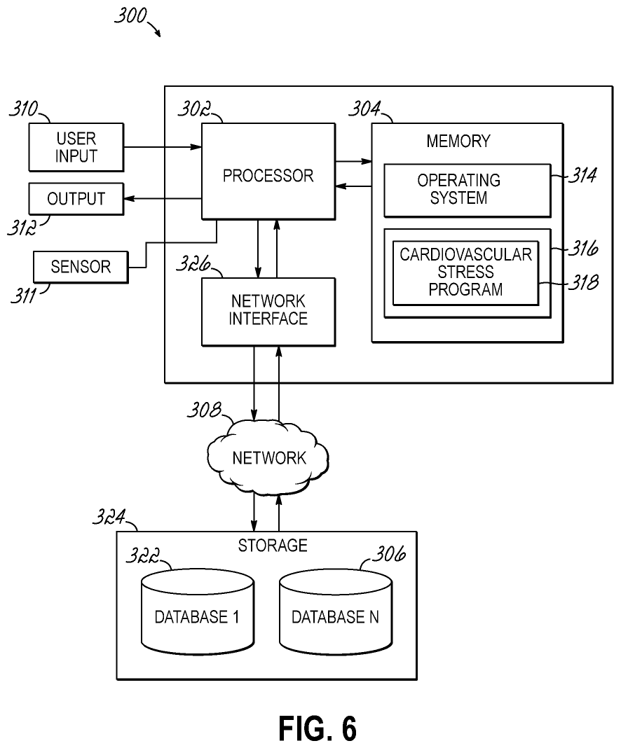

As noted herein, the invention may be implemented in a suitable computer device, such as a tablet device, such as the device used to implement the CVInsight® available from Intelomed, Inc. However, other devices or systems may be used. For example, FIG. 6 illustrates an exemplary hardware and software environment for an apparatus 300 suitable for implementing the process and system consistent with the invention. For the purposes of the invention, apparatus 300 may represent practically any computer, computer system, or programmable device, mobile device e.g., portable tablets and devices, handheld devices, network devices, mobile phones multi-user or single-user computers, desktop computers, etc. Apparatus 300 will hereinafter be referred to as a “computer” although it should be appreciated that the term “apparatus” may also include other suitable programmable electronic devices.

Computer 300 typically includes at least one processor 302 coupled to a memory 304. Processor 302 may represent one or more processors (e.g. microprocessors), and memory 304 may represent the random access memory (RAM) devices comprising the main storage of computer 300, as well as any supplemental levels of memory, e.g., cache memories, non-volatile or backup memories (e.g. programmable or flash memories), read-only memories, etc. In addition, memory 304 may be considered to include memory storage physically located elsewhere in computer 300, e.g., any cache memory in a processor 302, as well as any storage capacity used as a virtual memory, e.g., as stored on a mass memory storage device or system 306 or another computer that may be coupled to computer 300, such as through a network 308. The mass storage system 306 may include cloud storage or other appropriate remote storage components that may support one or more (1−n) databases 322, 324. The mass storage system might be accessed through network 308.

Computer 300 also typically receives a number of inputs and outputs for communicating information externally. For interface with a user or operator, computer 10 typically includes one or more user input devices 310 (e.g., a touchscreen, camera, microphone, keyboard, mouse, trackball, joystick, keypad, stylus, among others). In accordance with one embodiment of the invention, the input device is a sensor 311 for detecting a biological signal, such as a PPG signal. Computer 300 may also include one or more output or display elements 312 (e.g., a screen, separate monitor or display, speaker, among others). The interface to computer 300 may also be through an external terminal connected directly or remotely to computer 300, or through another computer communicating with computer 300, such as via a network 308.

Computer 300 operates under the control of an operating system 314, and executes or otherwise relies upon various computer software applications, components, programs, objects, modules, data structures, etc 316. (e.g. Respiratory Stress application 318). Application 318, for example, may provide the various processing steps set forth herein for characterizing respiratory stress. Computer 300 communicates on the network 308 through a suitable wired or wireless network interface 326, such as a WiFi link. The computer 300 may be coupled with appropriate storage through network interface 326 and network 308.

In general, the routines executed to implement the embodiments of the invention, whether implemented as part of an operating system or a specific application, component, program, object, module or sequence of instructions will be referred to herein as “computer program code”, or simply “program code”. The computer program code typically comprises one or more instructions that are resident at various times in various memory and storage devices in a computer, and that, when read and executed by one or more processors or processing units in a computer, causes that computer to perform the steps necessary to execute steps or elements embodying the desired functionality of various aspects of the invention. Moreover, while the invention has and hereinafter will be described in the context of fully functioning computers and computer systems, those skilled in the art will appreciate that the various embodiments of the invention are capable of being distributed as a program product in a variety of forms. The invention applies equally regardless of the particular type of computer readable storage media used to actually carry out the distribution, e.g., physical, recordable type storage media such as volatile and non-volatile memory devices, portable or thumb drives and various disks, or remote storage, such as on a server or cloud storage, that may be accessed via a network connection. Furthermore, since functionality of the system might be distributed between various components, such as servers, mobile devices and other components, the invention is not limited to specific components handling specific functions.

Such computer readable media may include computer readable storage media and communication media. Computer readable storage media is non-transitory in nature, and may include volatile and non-volatile, and removable and non-removable media implemented in any method or technology for storage of information, such as computer-readable instructions, data structures, program modules or other data. Computer readable storage media may further include RAM, ROM, erasable programmable read-only memory (EPROM), electrically erasable programmable read-only memory (EEPROM), flash memory or other solid state memory technology, CD-ROM, DVD, or other optical storage, magnetic cassettes, magnetic tape, magnetic disk storage or other magnetic storage devices, or any other medium that can be used to store the desired information and which can be accessed by a computer or other device. Communication media may embody computer readable instructions, data structures or other program modules. By way of example, and not limitation, communication media may include wired media such as a wired network or direct-wired connection, and wireless media such as acoustic, RF, infrared and other wireless media. Combinations of any of the above may also be included within the scope of computer readable media.

In addition, various program code described hereinafter may be identified based upon the application or software component within which it is implemented in specific embodiments of the invention. However, it should be appreciated that any particular program nomenclature that follows is merely for convenience, and thus the invention should not be limited to use solely in any specific application identified and/or implied by such nomenclature. Furthermore, given the typically endless number of manners in which computer programs may be organized into routines, procedures, methods, modules, objects, and the like, as well as the various manners in which program functionality may be allocated among various software layers that are resident within a typical computer (e.g., operating systems, libraries, APIs, applications, applets, etc.), it should be appreciated that the invention is not limited to the specific organization and allocation of program functionality described herein.

Those skilled in the art will recognize that the exemplary environment illustrated in FIG. 6 is not intended to limit the present invention. Indeed, those skilled in the art will recognize that other alternative hardware and/or software environments may be used without departing from the scope of the invention.

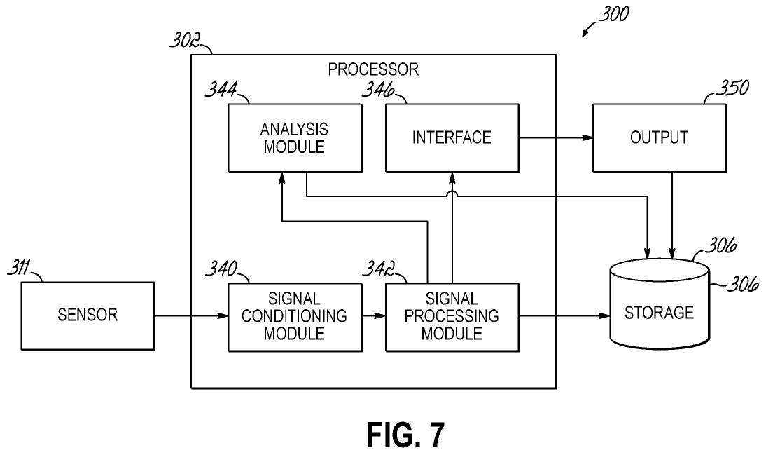

FIGS. 7-10 illustrate various other possible and non-limiting embodiments of a computer system 300 in which embodiments of the present invention may be implemented. Various embodiments of a system 300 for detecting respiratory stress include a sensor 311 that acquires a biological signal, a processor 302 that includes one or more appropriate software modules 340, 342, 344 residing in memory for processing and analyzing the sensor signal, and an interface module 346 that generates appropriate output parameter 350, as discussed herein. In the embodiment illustrated in 6, the sensor 311 is in communication with, via a wireline or wireless connection, the processor 302 that is external to the sensor 10. The system 300 may further include appropriate memory storage 306, as noted, that might be in the computer system or device or remotely coupled through a network interface (not shown in FIG. 6).

As described herein, the sensor for detecting a biological signal may be any invasive or non-invasive device that includes appropriate circuitry to acquire a biological signal.

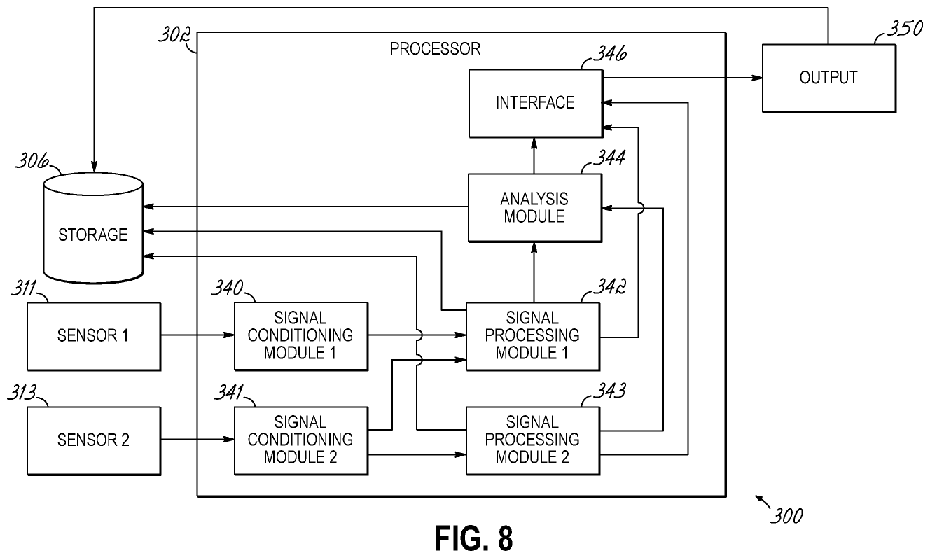

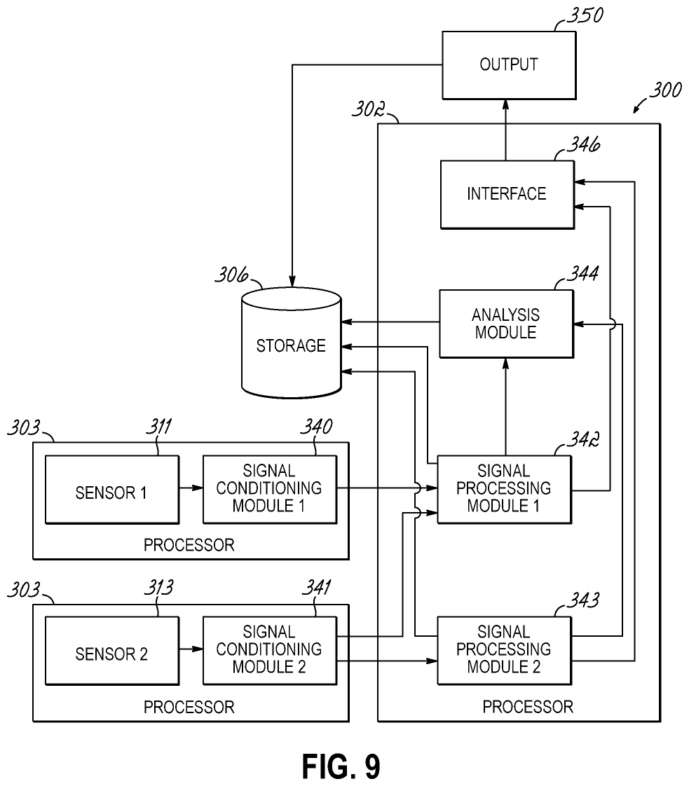

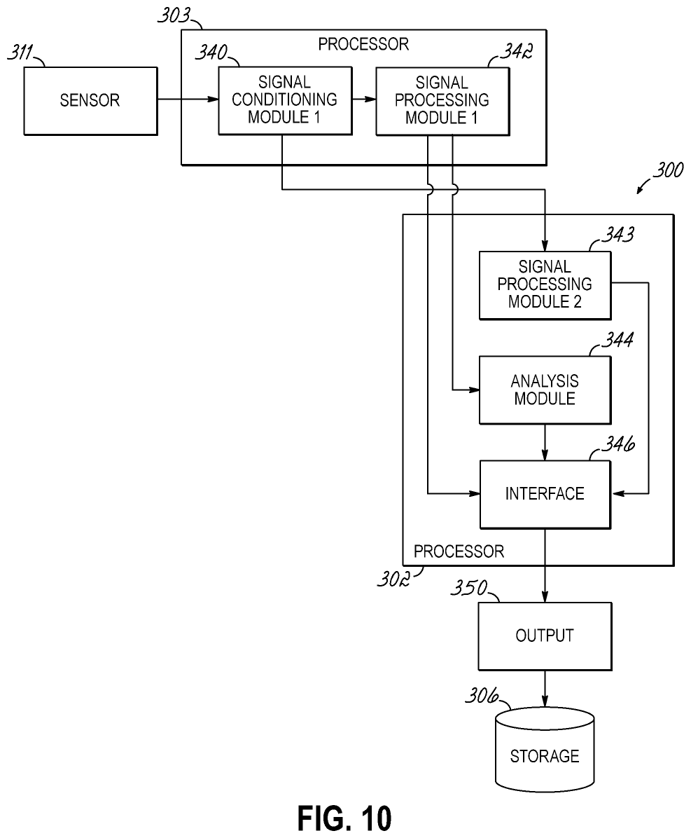

Although FIG. 7 illustrates the case of a processor 302, it can be understood that in various embodiments, the system may include one or more second processors 303, as illustrated in FIGS. 9-10. As illustrated in FIGS. 8-9, the second processor 303 may be external to the first processor 302 and optionally may be located within the first sensor 311, and may include at least one module 342 configured for post-acquisition processing of the first signal and that communicates, via a wired or wireless connection, with the first processor 302 for further processing of the signal prior to generation of an output 350.

Although FIG. 6 illustrates the case of a first sensor 311 it can be understood that the system 300 may include at least one second sensor 313 configured to record at least one second signal, as shown in FIGS. 8-9. In various embodiments, as illustrated, the second sensor 313 communicates with the first processor 302, via a wired or wireless connection, to transmit the second signal to the first processor 302 for post-acquisition processing and analysis by modules 340, 342, 344 collocated therein. In various embodiments, the second sensor 313 may include a second processor 303 that includes at least one module 341, 343, configured to process the acquired signal.

Table 4 provides a list of examples of various primary signal captured from each that might be used for capturing a biological signal for use with the present invention. This list is exemplary only and is not intended to be inclusive.

TABLE 4
Primary Sensors and Primary Signals.

| Primary Sensor | Primary Signal Acquired |
|---|---|
| Photo-optic sensor (transmissive) | Blood density |
| Photo-optic sensor (reflective) | Blood density |
| Pressure transducer | Pulse pressure |
| Tonometry device | Vascular pressure |
| Strain gauge | Vessel circumference |
| Ultrasound device | Vessel diameter |
| Electrical impedance | Fluid electrical conductivity |
| Radar device | Cardiac pulses |
| Non-contact PPG (camera) | Images |

In one embodiment, the primary sensor 311 is a photo-optic sensor that acquires a photo-optic signal as described above. The photo-optic sensor may acquire the signal at a wavelength at which density changes reflect changes in density of both oxygenated and deoxygenated blood. For example, in one embodiment, the photo-optic sensor acquires the signal at wavelengths between about 700 nm and about 950 nm.

The photo-optic sensor may be either transmissive or reflective. In various embodiments, the photo-optic sensor is a reflective photo-optic sensor. The transmitter and the receiver are separated by a distance. In embodiments, the reflective photo-optic sensor is positioned on a patient's forehead or other well-vascularized skin surfaces. In other various embodiments, the photo-optic sensor is a transmissive photo-optic sensor. In embodiments, the transmissive photo-optic sensor is positioned on a patient's finger or the like and light is transmitted through the finger or the like to a receiver on the other side of the finger.

In another embodiment, the sensor is a pressure transducer that acquires a pulse pressure signal that indicates pulsatile changes in total blood volume. In embodiments, the pressure transducer is non-invasive. In other embodiments, the pressure transducer receives the pulse pressure signal from an arterial pressure line implanted in an artery.

In another embodiment, the sensor is a tonometry device that acquires a signal that measures changes in vascular tension or pressure that result from changes in blood density that occur as the pulse wave travels through the arterial bed. In embodiments, tissue is applanated to obtain the vascular pressure change.

In another embodiment, the primary sensor is a strain gauge that acquires a signal that measures changes in the circumference of an extremity that result from changes in blood density that occur as the pulse wave travels through the arterial bed.

In another embodiment, the primary sensor is an ultrasound device that acquires a signal that measures changes in the diameter of a blood vessel that result from changes in blood density that occur as the pulse wave travels through the arterial bed.

In another embodiment, the primary sensor is an electrical impedance device that acquires a signal that measures changes in electrical conductivity of the blood that result from changes in blood density that occur as the pulse wave travels through the arterial bed.

In another embodiment, the primary sensor is a radar device that acquires a signal that measures changes in contraction of the cardiac muscle during a cardiac cycle.

In another embodiment, the primary sensor is a camera that acquires a signal that measures changes in color, reflecting the changes in blood density that occur during each cardiac cycle.

While the present invention has been illustrated by the description of the embodiments thereof, and while the embodiments have been described in considerable detail, it is not the intention of the Applicant to restrict or in any way limit the scope of the appended claims to such detail. Additional advantages and modifications will readily appear to those skilled in the art. Therefore, the invention in its broader aspects is not limited to the specific details representative apparatus and method, and illustrative examples shown and described. Accordingly, departures may be made from such details without departure from the spirit or scope of Applicant's general inventive concept.

What is claimed is:

1. A method of evaluating the cardiovascular health of a patient comprising:
obtaining at least one biological signal from a patient in real time, the biological signal having a waveform;
using at least one processor, processing the waveform of the biological signal and deriving at least one perfusion parameter for the patient;
determining a baseline value for the at least one perfusion parameter;
monitoring, in real time, the derived perfusion parameter and determining a variation of the computed perfusion parameter from the baseline value;
evaluating the determined variation of the derived perfusion parameter with respect to at least one variation threshold;
measuring a duration of time associated with the determined variation that is reflective of the amount of time the derived perfusion parameter variation is above or below the at least one variation threshold;
based on the evaluation of the derived perfusion parameter with respect to the at least one variation threshold and the measured duration of time, determining a cardiovascular stress index for the patient;
displaying the cardiovascular stress index for alerting a caregiver to the cardiovascular health of a patient.

2. The method of claim 1 further comprising processing the waveform in at least one of the time domain or the frequency domain for computing the at least one perfusion parameter.

3. The method of claim 1 wherein the at least one biological signal is a photoplethysmogram signal.

4. The method of claim 1 wherein the perfusion parameter includes a pulse amplitude that is determined in the time domain.

5. The method of claim 1 wherein the perfusion parameter includes a pulse strength that is determined in the frequency domain.

6. The method of claim 1 wherein the perfusion parameter variation includes a percentage decrease of the perfusion parameter from the baseline value for the perfusion parameter.

7. The method of claim 1 wherein the variation threshold includes at least one of a 20% decrease in the perfusion parameter from a baseline value or a 40% decrease in perfusion parameter from a baseline value.

8. The method of claim 1 further comprising comparing a measured duration of time associated with the determined variation against a threshold and using that measured duration of time against the threshold for determining a cardiovascular stress index for the patient.

9. The method of claim 8 wherein the time duration threshold includes at least one of 30 minutes, 60 minutes, 120 minutes, 180 minutes.

10. A system comprising:
a sensor device configured for operative communication with a patient to generate at least one biological signal, having a waveform curve;
a device with at least one processor, the device configured for being coupled with the sensor;
program code configured to be executed on the at least one processor, the program code causing the processor of the device to:
process the waveform of the biological signal and derive at least one perfusion parameter for the patient, to determine a baseline value for the at least one perfusion parameter, to monitor, in real time, the derived perfusion parameter and determine a variation of the computed perfusion parameter from the baseline value, to evaluate the determined variation of the derived perfusion parameter with respect to at least one variation threshold, to measure a duration of time associated with the determined variation that is reflective of the amount of time the derived perfusion parameter variation is above or below the at least one variation threshold;
to determine a cardiovascular stress index for the patient, based on the evaluation of the derived perfusion parameter with respect to the at least one variation threshold and the measured duration of time;
the device configured with a display for displaying the respiratory stress metric to a user.

11. The system of claim 10 wherein the program code is configured processing the waveform in at least one of the time domain or the frequency domain for computing the at least one perfusion parameter.

12. The system of claim 10 wherein the at least one biological signal is a photoplethysmogram signal.

13. The system of claim 10 wherein the perfusion parameter includes a pulse amplitude that is determined in the time domain.

14. The system of claim 10 wherein the perfusion parameter includes a pulse strength that is determined in the frequency domain.

15. The system of claim 10 wherein the perfusion parameter variation includes a percentage decrease of the perfusion parameter from the baseline value for the perfusion parameter.

16. The system of claim 10 wherein the program code is configured for generating the respiration rate waveform curve with a B-spline curve fit analysis.

17. The system of claim 13 wherein the variation threshold includes at least one of a 20% decrease in the perfusion parameter from a baseline value or a 40% decrease in perfusion parameter from a baseline.

18. The system of claim 10 further comprising comparing a measured duration of time associated with the determined variation against a threshold and using that measured duration of time against the threshold for determining a cardiovascular stress index for the patient.

19. The system of claim 10 wherein the time duration threshold includes at least one of 30 minutes, 60 minutes, 120 minutes, 180 minutes.
